(19) United States
(12) Patent Application Publication
(10) Pub. No.: US 2010/0203102 A1
(43) Pub. Date: Aug. 12, 2010

(54) COMPOSITIONS AND METHODS FOR TREATING POST-OPERATIVE PAIN USING BUPIVACAINE AND AN ANTI-ONFLAMMATORY AGENT

(75) Inventor: Amira Wohabrebbi, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/423,201

(22) Filed: Apr. 14, 2009

Related U.S. Application Data
(60) Provisional application No. 61/151,390, filed on Feb. 10, 2009.

(51) Int. Cl. A61F 2/00; A61K 31/445

(52) U.S. Cl. 424/426; 514/330

(57) ABSTRACT

Effective treatments of pain that accompanies post-operative surgeries are provided. Through the administration of an effective amount of a combination of bupivacaine and an anti-inflammatory agent at or near a target site, one can alleviate or prevent pain while preventing undesirable levels of inflammation. This administration of bupivacaine and an anti-inflammatory agent is particularly useful following surgery.

This application claims the benefit of the filing date of U.S. Provisional Patent Application No. 61/151,390 filed Feb. 10, 2009, the entire disclosure of which is hereby incorporated by reference into the present disclosure.

### BACKGROUND

Pain is typically experienced when the free nerve endings of pain receptors are subject to mechanical, thermal, chemical or other noxious stimuli. These pain receptors can transmit signals along afferent neurons to the central nervous system and then to the brain. When a person feels pain, any one or more of a number of problems can be associated with this sensation, including but not limited to reduced function, reduced mobility, complication of sleep patterns, and decreased quality of life.

The causes of pain include inflammation, injury, disease, muscle spasm and the onset of a neuropathic event or syndrome. By way of example, inflammatory pain can occur when tissue is damaged, as can result from surgery or an adverse physical, chemical or thermal event or from infection by a biologic agent. When a tissue is damaged, a host of endogenous pain inducing substances, for example, bradykinin and histamine can be released from the injured tissue. The pain inducing substances can bind to receptors on the sensory nerve terminals and thereby initiate afferent pain signals. After activation of the primary sensory afferent neurons, the projection neurons may be activated. These neurons carry the signal via the spinothalamic tract to higher parts of the central nervous system. Inflammatory pain is generally reversible and may subside when the injured tissue has been repaired or the pain inducing stimuli is removed.

When a patient undergoes surgery, there is an increased likelihood that absent the use of analgesics, pain will be felt during and/or after surgery. Thus, this pain, including the post-operative pain is to a degree predictable with respect to whom it most likely will affect, is most likely to occur within a finite window of time, and is localized to a site at or near the site of a surgical procedure.

One known class of pharmaceuticals to treat post-operative pain is opiods. This class of compounds is well-recognized as being among the most effective type of drugs for controlling post-operative pain. Unfortunately, because opiods are administered systemically, the associated side effects raise significant concerns, including disabling the patient, depressing the respiratory system, constipation, and psychoactive effects such as sedation and euphoria, thereby instituting a hurdle to recovery and regained mobility. Further, because of these side-effects, physicians typically limit the administration of opiods to within the first twenty-four hours post-surgery. Thus, it would be preferable to use non-narcotic drugs that deliver direct, localized pain control at a surgical site.

One pharmaceutical that is known to the medical profession is bupivacaine, which is widely recognized as a local anesthetic for infiltration, nerve block, epidural and intrathecal administration. In general, bupivacaine, also referred to as 1-butyl-N-(2,6-dimethylphenyl) piperidine-2-carboxamide (C₁₈H₂₈N₂O) may be represented by the following structure:

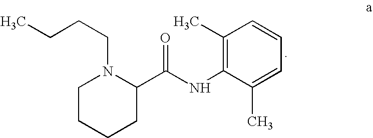

Bupivacaine is now known commonly administered to patients in order to treat pain, such as post-operative pain. However, it has been observed that following administration of bupivacaine, there is often an unacceptable level of inflammation. This inflammation can have deleterious effects on an individual.

Because the risks associated with this inflammation need to balanced with the beneficial effects of bupivacaine, there is a need for effective administration of bupivacaine in which the amount of inflammation can be reduced.

### SUMMARY

Compositions are provided comprising bupivacaine in combination with one or more anti-inflammatory agents that are administered in order to relieve pain after surgery. Methods for administering these compositions are also provided. When administered in an effective amount, particularly in sustain release formulations, the compositions and methods may provide effective treatments for post-operative pain that reduce the amount of inflammation that accompanies the administration of bupivacaine.

According to one embodiment there is a drug depot comprising: (a) a therapeutically effective amount of bupivacaine; and (b) a therapeutically effective amount of an anti-inflammatory agent.

According to another embodiment there is a method of treating or preventing postoperative pain with an avoidance of an unacceptable amount of inflammation, the method comprising administering a therapeutically effective amount of bupivacaine and an anti-inflammatory agent to a target tissue site beneath the skin, wherein the drug depot releases an effective amount of the bupivacaine the anti-inflammatory agent over a period of 3 to 14 days or 5 to 12 days or 7 to 10 days.

According to another embodiment there is an implantable drug depot useful for localized delivery to a site beneath the skin of a patient, the drug depot comprising: a therapeutically effective amount of bupivacaine and an anti-inflammatory agent, wherein the drug depot is capable of releasing the bupivacaine and the anti-inflammatory agent over three days to fourteen days where the depot is capable of releasing a first percentage of bupivacaine relative to a total amount of bupivacaine over the first two days and a first percentage of anti-inflammatory agent relative to a total amount of anti-inflammatory agent over the first two days, wherein the first percentage of anti-inflammatory agent is less than the first percentage of bupivacaine.

According to another embodiment, there is a method of inhibiting postoperative pain, the method comprising delivering one or more biodegradable drug depots comprising a therapeutically effective amount of bupivacaine thereof and an anti-inflammatory agent to a target tissue site beneath the skin before, during or after surgery, wherein the drug depot releases an effective amount of bupivacaine and the anti-inflammatory agent over a period of 3 to 14 days or 5 to 12 days or 7 to 10 days.

According to another embodiment there is an implantable drug depot useful for preventing or treating postoperative pain in a patient in need of such treatment, the implantable drug depot comprising a therapeutically effective amount of bupivacaine and an anti-inflammatory agent, the drug depot being implantable at a site beneath the skin to prevent or treat postoperative pain, wherein the drug depot releases an effective amount of the bupivacaine and the anti-inflammatory agent over a period of 3 to 14 days or 5 to 12 days or 7 to 10 days.

According to another embodiment, there is an implantable drug depot useful for localized delivery to a site beneath the skin of a patient, the drug depot comprising: a therapeutically effective amount of bupivacaine and an anti-inflammatory agent, wherein the drug depot is capable of releasing the bupivacaine and the anti-inflammatory agent over three days to fourteen days where the depot is capable of releasing a first percentage of bupivacaine relative to a total amount of bupivacaine over the first two days and a first percentage of anti-inflammatory agent relative to a total amount of anti-inflammatory agent over the first two days, wherein the first percentage of bupivacaine is greater than the first percentage of the anti-inflammatory agent. Examples of anti-inflammatory agents include but are not limited to COX-2 inhibitors and NSAIDS.

According to another embodiment there is an implantable drug depot useful for localized delivery to a site beneath the skin of a patient, the drug depot comprising: a therapeutically effective amount of bupivacaine and an anti-inflammatory agent, wherein the drug depot is capable of releasing the bupivacaine and the anti-inflammatory agent over three days to fourteen days where the depot is capable of releasing a first percentage of bupivacaine relative to a total amount of bupivacaine over the first two days and a first percentage of anti-inflammatory agent relative to a total amount of anti-inflammatory agent over the first two days, wherein the first percentage of anti-inflammatory agent is greater than the first percentage of bupivacaine.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a number of common locations within a patient that may be sites at which surgery takes place and locations at which a drug depot containing bupivacaine and an anti-inflammatory agent can locally be administered thereto and used to treat post-operative pain.

FIG. 2 illustrates a schematic dorsal view of the spine and sites where the drug depot containing bupivacaine and the anti-inflammatory agent can locally be administered thereto.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

Additionally, unless otherwise specified or apparent from context, the following terms and phrases have the meanings provided below.

The term “biodegradable” includes that all or parts of the drug depot will degrade over time by the action of enzymes, by hydrolytic action and/or by other similar mechanisms in the human body. In various embodiments, “biodegradable” includes that the depot (e.g., microparticle, microsphere, etc.) can break down or degrade within the body to non-toxic components after or while a therapeutic agent has been or is being released. By “bioerodible” it is meant that the depot will erode or degrade over time due, at least in part, to contact with substances found in the surrounding tissue, fluids or by cellular action. By “bioabsorbable” it is meant that the depot will be broken down and absorbed within the human body, for example, by a cell or tissue. “Biocompatible” means that the depot will not cause substantial tissue irritation or necrosis at the target tissue site.

A “depot” includes but is not limited to capsules, microspheres, microparticles, microcapsules, microfibers particles, nanospheres, nanoparticles, coating, matrices, wafers, pills, pellets, emulsions, liposomes, micelles, gels, or other pharmaceutical delivery compositions or a combination thereof. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient” or “API.” It will be understood that unless otherwise specified, a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug depot provides an optimal drug concentration gradient of the therapeutic agent at a distance of up to about 0.01 cm to about 10 cm from the implant site, and comprises bupivacaine and an anti-inflammatory agent.

A “drug depot” of the present invention is the composition in which the bupivacaine or its pharmaceutically acceptable salts and/or the anti-inflammatory agent are administered to the body. Thus, these active ingredients may be combined in the same or different drug depots. A drug depot may be designed to have a physical structure to facilitate implantation and retention in a desired site (e.g., a disc space, a spinal canal, a tissue of the patient, particularly at or near a site of surgery, etc.). The drug depot may comprise a pump that holds and administers the pharmaceutical. In some embodiments, the drug depot has pores that allow release of the drug from the depot. The drug depot will allow fluid in the depot to displace the drug. However, cell infiltration into the depot will be prevented by the size of the pores of the depot. In this way, in some embodiments, the depot should not function as a tissue scaffold and does not allow tissue growth. Rather, the drug depot will solely be utilized for drug delivery. In some embodiments, the pores in the drug depot will be less than 250 to 500 microns. This pore size will prevent cells from infiltrating the drug depot and laying down scaffolding cells. Thus, in this embodiment, drug will elute from the drug depot as fluid enters the drug depot, but cells will be prevented from entering. In some embodiments, where there are little or no pores, the drug will elute out from the drug depot by the action of enzymes, by hydrolytic action and/or by other similar mechanisms in the human body.

The phrase “immediate release” is used herein to refer to one or more therapeutic agent(s) that is introduced into the body and that is allowed to dissolve in or become absorbed at the location to which it is administered, with no intention or mechanism of delaying or prolonging the dissolution or absorption of the drug.

“Localized” delivery includes, delivery where one or more drugs are deposited within a tissue, for example, a nerve root of the nervous system or a region of the brain, or in close proximity (within about 10 cm, or preferably within about 5 cm or about 1 cm, for example) thereto.

The term “mammal” refers to organisms from the taxonomy class “mammalian,” including but not limited to humans, other primates such as chimpanzees, apes, orangutans and monkeys, rats, mice, cats, dogs, cows, horses, etc.

The phrase “release rate profile” refers to the percentage of active ingredient that is released over fixed units of time, e.g., mcg/hr, mcg/day, 10% per day for ten days, etc. As persons of ordinary skill know, a release rate profile may be but need not be linear. By way of a non-limiting example, the drug depot may be a ribbon-like fiber that releases the bupivacaine and an anti-inflammatory agent over a period of time. By way of further non-limiting examples, the release can be continuous or pulse doses where the drug is released daily.

The phrases “sustained release” and “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the drug depot, or a matrix or component thereof, or as the result of metabolic transformation or dissolution of the therapeutic agent(s) or conjugates of therapeutic agent(s).

A “targeted delivery system” provides delivery of one or more drugs depots, gels or depots dispersed in the gel having a quantity of therapeutic agent that can be deposited at or near the target site as needed for treatment of pain, inflammation or other disease or condition. In various embodiments the formulations are preservative free.

A “therapeutically effective amount” or “effective amount” is such that when administered, the drug results in alteration of the biological activity, such as, for example, inhibition of inflammation, reduction or alleviation of pain or spasticity, or improvement in the condition through muscle relaxation, etc. The dosage administered to a patient can be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments the formulation is designed for immediate release. In other embodiments the formulation is designed for sustained release. In other embodiments, the formulation comprises one or more immediate release surfaces and one or more sustained release surfaces. For example, a bolus or immediate release formulation of bupivacaine and an anti-inflammatory agent may be placed at or near the surgery site and a sustain release formulation may also be placed at or near the same site. Thus, even after the bolus becomes completely accessible, the sustain release formulation would continue to provide the active ingredient for the intended tissue.

The terms “treating” or “treatment” in reference to a disease or condition refer to executing a protocol that may include administering one or more drugs to a patient (human, other normal or otherwise), in an effort to alleviate signs or symptoms of the disease. Alleviation can occur prior to signs or symptoms of the disease or condition appearing, as well as after their appearance. Thus, the terms “treating” and “treatment” include “preventing” or “prevention” of disease or undesirable condition. In addition, “treating” and “treatment” do not require complete alleviation of signs or symptoms, do not require a cure, and specifically include protocols that have only a marginal effect on the patient. By way of example, the administration of the effective dosages of bupivacaine and an anti-inflammatory agent may be used to prevent, treat or relieve the symptoms of pain incidental to surgery while preventing undesirable levels of inflammation.

The abbreviation “DLG” refers to poly(DL-lactide-co-glycolide).

The abbreviation “DL” refers to poly(DL-lactide).

The abbreviation “LG” refers to poly(L-lactide-co-glycolide).

The abbreviation “CL” refers to polycaprolactone.

The abbreviation “DLCL” refers to poly(DL-lactide-co-caprolactone).

The abbreviation “LCL” refers to poly(L-lactide-co-caprolactone).

The abbreviation “G” refers to polyglycolide.

The abbreviation “PEG” refers to poly(ethylene glycol).

The abbreviation “PLGA” refers to poly(lactide-co-glycolide) also known as poly(lactic-co-glycolic acid), which are used interchangeably.

The abbreviation “PLA” refers to polylactide.

The abbreviation “POE” refers to poly(orthoester).

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

Further, when referring to bupivacaine, unless otherwise specified or apparent from context it is understood that the inventors are also referring to pharmaceutically acceptable salts. Some examples of potentially pharmaceutically acceptable salts include those salt-forming acids and bases that do not substantially increase the toxicity of the compound. Some examples of these salts include salts of alkali metals such as magnesium, potassium and ammonium. Salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like. To the extent these salts of bupivacaine can be created for safe administration to a mammal, they are within the scope of the present invention.

Further, the bupivacaine may also be used in a base form.

The methods and compositions described herein are not limited to uses in connection with any specific surgery and include but are not limited to treatment of pain that may be associated with arthroscopic surgery, laparoscopic surgery, open back surgery, oral surgery, etc.

Anti-inflammatory agents that may be co-administered according to various embodiments of the present invention include, but are not limited to, salicylates, diflunisal, sulfasalazine [2-hydroxy-5-[-4-[C2-pyridinylamino)sulfonyl]azo]benzoic acid], indomethacin, ibuprofen, naproxen, ketorolac, tolmetin, or pharmaceutically acceptable salts thereof and diclofenac, ketoprofen, fenamates (mefenamic acid, meclofenamic acid), enolic acids (piroxicam, meloxicam), nabumetone, etodolac, nimesulide, apazone, gold, sulindac or tepoxalin; antioxidants, such as dithiocarbamate, steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone, and COX-2 inhibitors, including but not limited to celecoxib, rofecoxib, valdecoxib, etoricoxib, lumiracoxib, and meloxicam or a combination thereof.

The bupivacaine and anti-inflammatory agent may be administered with a muscle relaxant. Exemplary muscle relaxants include by way of example and not limitation, alcuronium chloride, atracurium bescylate, baclofen, carbolonium, carisoprodol, chlorphenesin carbamate, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The drug depot may also comprise other therapeutic agents or active ingredients in addition to the bupivacaine and an anti-inflammatory agent. These therapeutic agents, in various embodiments, block the transcription or translation of TNF-α or other proteins in the inflammation cascade. Suitable therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents include IL-1 inhibitors, such Kineret® (anakinra) which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α). It is contemplated that where desirable, a pegylated form of the above may be used. Examples of other therapeutic agents include NF kappa B inhibitors such as glucocorticoids, antioxidants, such as dithiocarbamate, and other compounds, such as, for example, sulfasalazine.

Additional specific examples of therapeutic agents suitable for use include, but are not limited to an analgesic agent, or osteoinductive growth factor or a combination thereof. Suitable anabolic growth or anti-catabolic growth factors include, but are not limited to, a bone morphogenetic protein, a growth differentiation factor, a LIM mineralization protein, CDMP or progenitor cells or a combination thereof.

Suitable analgesic agents include, but are not limited to, acetaminophen, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine amitriptyline, carbamazepine, gabapentin, pregabalin, or a combination thereof.

The bupivacaine and the anti-inflammatory may also be administered with non-active ingredients. These non-active ingredients may have multi-functional purposes including the carrying, stabilizing and controlling the release of the therapeutic agent(s). The sustained release process, for example, may be by a solution-diffusion mechanism or it may be governed by an erosion-sustained process. Typically, the depot will be a solid or semi-solid formulation comprised of a biocompatible material, which can be biodegradable. The term “solid” is intended to mean a rigid material, while, “semi-solid” is intended to mean a material that has some degree of flexibility, thereby allowing the depot to bend and conform to the surrounding tissue requirements.

In various embodiments, the non-active ingredients will be durable within the tissue site for a period of time equal to (for biodegradable components) or greater than (for non-biodegradable components) the planned period of drug delivery. For example, the depot material may have a melting point or glass transition temperature close to or higher than body temperature, but lower than the decomposition or degradation temperature of the therapeutic agent. However, the pre-determined erosion of the depot material can also be used to provide for slow release of the loaded therapeutic agent(s).

In various embodiments, the drug depot may not be biodegradable. For example, the drug depot may comprise polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. Typically, these drug depots may need to be removed after a certain period of time.

In some instance, it may be desirable to avoid having to remove the drug depot after use. In those instances, the depot may comprise a biodegradable material. There are numerous materials available for this purpose that have the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

In various embodiments, the depot may comprise a bioerodible, a bioabsorbable, and/or a biodegradable biopolymer that may provide immediate release, or sustained release of the bupivacaine and the anti-inflammatory agent. Examples of suitable sustain release biopolymers include but are not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. As persons of ordinary skill are aware, mPEG may be used as a plasticizer for PLGA, but other polymers/excipients may be used to achieve the same effect. mPEG imparts malleability to the resulting formulations.

The polymers may be processed by either solvent or heat as long as the formulation containing drug and/or excipient is well mixed within the dosage form. Excipients may be added to the formulation to help with the drug release properties and/or to help with the mechanical properties of the polymer. For example, adding mPEG to PLGA has a plasticizing effect on the polymer, but it also effects the diffusion properties of the drug from the polymer.

The depot may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfite, sodium bisulfate, sodium thiosulfate, thimerosal, methylparaben, polyvinyl alcohol and phenylethyl alcohol; solubility adjusting agents; stabilizers; and/or cohesion modifiers. Typically, any such inactive materials will be present within the range of 0-75 wt. %, and more typically within the range of 0-30 wt. %. If the depot is to be placed in the spinal area, in various embodiments, the depot may comprise sterile preservative free material.

The depot can be different sizes, shapes and configurations. There are several factors that can be taken into consideration in determining the size, shape and configuration of the drug depot. For example, both the size and shape may allow for ease in positioning the drug depot at the target tissue site that is selected as the implantation or injection site. In addition, the shape and size of the system should be selected so as to minimize or prevent the drug depot from moving after implantation or injection. In various embodiments, the drug depot can be shaped like a sphere, a cylinder such as a rod or fiber, a flat surface such as a disc, film or sheet (e.g., ribbon-like) and the like. Flexibility may be a consideration so as to facilitate placement of the drug depot. In various embodiments, the drug depot can be different sizes, for example, the drug depot may be a length of from about 0.5 mm to 5 mm and have a diameter of from about 0.01 mm to about 2 mm. In various embodiments, the drug depot may have a layer thickness of from about 0.005 mm to 1.0 mm, such as, for example, from 0.05 to 0.75 mm.

In various embodiments, when the drug depot comprises a ribbon-like fiber, it may be placed at the incision site before the site is closed. The ribbon-like strips may for example be made of thermosplastic materials. Additionally, specific materials that may be advantageous for use as ribbon-like strips include but are not limited to the compounds identified above as sustain release biopolymers. The ribbon-like strip may be formed by mixing the bupivacaine and the anti-inflammatory agent with a polymer and then extruding it.

Radiographic markers can be included on the drug depot to permit the user to position the depot accurately into the target site of the patient. These radiographic markers will also permit the user to track movement and degradation of the depot at the site over time. In this embodiment, the user may accurately position the depot in the site using any of the numerous diagnostic imaging procedures. Such diagnostic imaging procedures include, for example, X-ray imaging or fluoroscopy. Examples of such radiographic markers include, but are not limited to, barium, calcium phosphate, and/or metal beads or particles. In various embodiments, the radiographic marker could be a spherical shape or a ring around the depot.

In various embodiments, the drug depot can be designed to cause an initial burst dose of therapeutic agent within the first twenty-four hours after implantation. “Initial burst” or “burst effect” or “bolus dose” refers to the release of therapeutic agent from the depot during the first twenty-four to forty-eight hours after the depot comes in contact with an aqueous fluid (e.g., synovial fluid, cerebral spinal fluid, etc.). The “burst effect” is believed to be due to the increased release of therapeutic agent from the depot. In alternative embodiments, the depot (e.g., gel) is designed to avoid this initial burst effect.

In various embodiments, the release profiles of bupivacaine and the anti-inflammatory agent are the same. In other embodiments, the release profile of bupivacaine and the anti-inflammatory agent are different. For example, because inflammation may be greater immediately post-surgery, in various embodiments, it may be advantageous for there to be a greater release of the anti-inflammatory agent at that time, i.e., a burst effect for the anti-inflammatory agent and either no burst effect for the bupivacaine or a relatively smaller burst effect for the bupivacaine relative to the anti-inflammatory agent. Thus, in some embodiments, there may be a release profile of the bupivacaine that releases a first percentage of the bupivacaine relative to the total amount of bupivacaine over the first two days and a release profile of the anti-inflammatory agent that releases a first percentage of the anti-inflammatory relative to the total amount of anti-inflammatory over the first two days. The percentage of anti-inflammatory released over those first two days may be greater than the percentage of bupivacaine released over those first two days. Thus, in some embodiments, more than 30 percent of the anti-flammatory is released in the first two days and less than 25 percent of the bupivacaine is released in that time period. In some embodiments, more than 40 percent of the anti-flammatory is released in the first two days and less than 35 percent of the bupivacaine is released in that time period. In some embodiments, more than 50 percent of the anti-flammatory is released in the first two days and less than 45 percent of the bupivacaine is released in that time period.

In other embodiments, it may be advantageous for there to be a greater release of the bupivacaine at that time, i.e., a burst effect for the bupivacaine agent and either no burst effect for the anti-inflammatory agent or a relatively smaller burst effect for the anti-inflammatory agent relative to the bupivacaine. Thus, in some embodiments, there may be a release profile of the bupivacaine that releases a first percentage of the bupivacaine relative to the total amount of bupivacaine over the first two days and a release profile of the anti-inflammatory agent that releases a first percentage of the anti-inflammatory agent relative to the total amount of anti-inflammatory agent over the first two days. The percentage of anti-inflammatory agent released over those first two days may be less than the percentage of bupivacaine released over those first two days. Thus, in some embodiments, more than 30 percent of the bupivacaine is released in the first two days and less than 25 percent of the anti-inflammatory agent is released in that time period. In some embodiments, more than 40 percent of the bupivacaine is released in the first two days and less than 35 percent of the anti-inflammatory agent is released in that time period. In some embodiments, more than 50 percent of the bupivacaine is released in the first two days and less than 45 percent of the anti-inflammatory agent is released in that time period.

Other exemplary release profiles of bupivacaine are described in co-pending application Compositions and Methods For Treating Post-Operative Pain Using Clonidine and Bupivacaine, attorney docket, P0031333.00, the contents of which are incorporated by reference herein.

In various embodiments the anti-inflammatory is ketoralac and the drug depot may release 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 75 mg, 80 mg, 85 mg, 90 mg, 95 mg, 100 mg, 105 mg, 110 mg, 115 mg, 120 mg, 125 mg, 130 mg, 135 mg, or 140 mg of ketorolac per day for a total of 3 to 10 days, or 3 to 5 days. In various embodiments, the drug depot may release 0.1 mg to 6 mg of ketorolac per hour for a total of 3 to 10 days, or 3 to 5 days to reduce, treat or prevent postoperative pain. In various embodiments, the drug depot releases 5%, 10%, 15%, 20%, 25%, 30%, 40%, 50%, 60%, 70%, 80%, 90%, 95%, or 99% of the ketorolac over a period of 3 to 10 days after the drug depot is administered to the target tissue site or 5 to 7 days.

In various embodiments, when there are two separate drug depots that are coadministered, the drug depot that contains the ketorolac may comprise from about 2.5% to 60% by weight ketorolac, from about 20% to 95% by weight PLGA, 5% to 30% by weight of mPEG. The ester form of ketorolac being more hydrophobic may, in various embodiments, provide a better release profile.

In various embodiments, the anti-inflammatory is sulindac. When referring to sulindac, unless otherwise specified or apparent from context it is understood that the inventors are also referring to pharmaceutically acceptable salts, pharmacologically-active derivatives of the sulindac or an active metabolite of the sulindac. As used herein, “pharmaceutically acceptable salts” refer to derivatives of the disclosed compounds (e.g., esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid. Pharmaceutically acceptable also includes the racemic mixtures ((+)-R and (−)-S enantiomers) or each of the dextro and levo isomers of the sulindac individually. The sulindac may be in the free acid or base form or be pegylated for long acting activity.

One well-known commercially available salt for sulindac is its sodium salt (e.g., available from Spectrum Chemical) or sulfide salt.

In one embodiment of the present invention, the sulindac and the dosage is from approximately 0.001 μg/day to approximately 100 mg/day. Additional dosages of sulindac can include from approximately 0.001 μg/day to approximately 200 mg/day; approximately 0.001 μg/day to approximately 100 mg/day; approximately 0.001 μg/day to approximately 1 mg/day; approximately 0.001 μg/day to approximately 500 μg/day; approximately 0.001 μg/day to approximately 100 μg/day; approximately 0.025 to approximately 75 μg/day; approximately 0.025 μg/day to approximately 65 μg/day; approximately 0.025 μg/day to approximately 60 μg/day; approximately 0.025 μg/day to approximately 55 μg/day; approximately 0.025 μg/day to approximately 50 μg/day; approximately 0.025 μg/day to approximately 45 μg/day; approximately 0.025 μg/day to approximately 40 μg/day; approximately 0.025 μg/day to approximately 35 μg/day; approximately 0.005 to approximately 30 μg/day; approximately 0.005 to approximately 25 μg/day; approximately 0.005 μg/day to approximately 20 μg/day; and approximately 0.005 μg/day to approximately 15 μg/day. In another embodiment, the dosage of sulindac is from approximately 0.01 μg/day to approximately 15 μg/day. In another embodiment, the dosage of sulindac is from approximately 0.01 μg/day to approximately 10 μg/day. In another embodiment, the dosage of sulindac is from approximately 0.01 μg/day to approximately 5 μg/day. In another embodiment, the dosage of sulindac is from approximately 0.01 μg/day to approximately 20 μg/day. In another embodiment, sulindac is administered in a drug depot that releases 9.6 μg/day.

In various embodiments, the anti-inflammatory is sulfasalazine. When referring to sulfasalazine, unless otherwise specified or apparent from context it is understood that the inventors are also referring to pharmaceutically acceptable salts or pharmacologically-active derivatives of the sulfasalazine or an active metabolite of the sulfasalazine. In the context of the present specification, unless otherwise stated, a pharmaceutically acceptable derivative of sulfasalazine means a pharmaceutically acceptable ester, salt or solvate of sulfasalazine or a pharmaceutically acceptable solvate of such an ester or salt. Examples of suitable esters of sulfasalazine include lower alkyl (C₁-C₆ alkyl) esters. Pharmaceutically acceptable salts include acid addition salts derived from pharmaceutically acceptable inorganic and organic acids such as a chloride, bromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, gluconate, tricarballylate, bydroxynaphthalene-carboxylate or oleate salt; or salts prepared from pharmaceutically acceptable inorganic and organic bases. Salts derived from inorganic bases include aluminium, ammonium, calcium, copper, ferric, ferrous, lithium, magnesium, manganic, manganous, potassium, sodium, zinc or bismuth salts. Salts derived from pharmaceutically acceptable organic bases include salts of primary, secondary and tertiary amines, cyclic amines like arginine, betaine, choline or the like. Examples of pharmaceutically acceptable solvates include hydrates.

The preparation of sulfasalazine is described, for example, in U.S. Pat. No. 2,396,145 and by Doraswamy, Guha, J. Indian. Chem. Soc., 23, 278 (1946). Pharmaceutically acceptable derivatives of sulfasalazine may be prepared by methods conventional in the art. Sulfasalazine may be obtained from Spectrum Chemical. Sulfasalazine may be capable of existing in stereoisomeric forms. It will be understood that sulfasalazine encompasses all geometric and optical isomers of the active ingredients and mixtures thereof including racemates, tautomers or mixtures thereof

Further, when referring to sulfasalazine the active ingredient may not only be in the salt form, but also in the free acid or base form (e.g., free acid).

In one embodiment, the dosage of sulfasalazine is from approximately 0.005 μg/day to approximately 3000 mg/day. Additional dosages of sulfasalazine include from approximately 0.005 μg/day to approximately 2000 mg/day; approximately 0.005 μg/day to approximately 1000 mg/day; approximately 0.005 μg/day to approximately 100 mg/day; approximately 0.005 μg/day to approximately 1 mg/day; approximately 0.005 μg/day to approximately 80 μg/day; approximately 0.01 μg/day to approximately 70 μg/day; approximately 0.01 μg/day to approximately 65 μg/day; approximately 0.01 μg/day to approximately 60 μg/day; approximately 0.01 μg/day to approximately 55 μg/day; approximately 0.01 μg/day to approximately 50 μg/day; approximately 0.01 μg/day to approximately 45 μg/day; approximately 0.01 to approximately 40 μg/day; approximately 0.025 μg/day to approximately 35 μg/day; approximately 0.025 μg/day to approximately 30 μg/day; approximately 0.025 μg/day to approximately 25 μg/day; approximately 0.025 μg/day to approximately 20 μg/day; and approximately 0.025 μg/day to approximately 15 μg/day. In another embodiment, the dosage of sulfasalazine is from approximately 0.05 μg/day to approximately 15 μg/day. In another embodiment, the dosage of sulfasalazine is from approximately 0.05 to approximately 10 μg/day.

In order to have different release profiles, one may create and implant separate depots, each of which contain one of the two active ingredients, or one may create depots that contain both ingredients, but the relative distance of the ingredients to the surface of the depots and/or the polymers associated with each active ingredient is different such that it causes the different release profiles. It is also noted that for any of the ranges provide above, those ranges are averages, and in some embodiments it may be that a greater release rate occurs in the first two days and a smaller rate occurs over the remaining days.

Additionally, the relative amounts of the bupivacaine and the anti-inflammatory agents may differ. For example, in some embodiments, there is a formulation in which there is a loading of bupivacaine of 50-80% and a loading of the anti-inflammatory of 1-40% by weight of the formulation. In some embodiments, there is a formulation in which there is a loading of bupivacaine of 50-60%, 60-70% or 70-80%. In some embodiments, there is a loading of anti-inflammatory of 1-10%, 10-20%, 20-30%, or 30-40% by weight of the formulation. In various embodiments, when the depot is a gel, the gel has a pre-dosed viscosity in the range of about 1 to about 500 centipoise (cps), 1 to about 200 cps, or 1 to about 100 cps. After the gel is administered to the target site, the viscosity of the gel will increase and the gel will have a modulus of elasticity (Young's modulus) in the range of about 1×10⁴ to about 6×10⁵ dynes/cm², or 2×10⁴ to about 5×10⁵ dynes/cm², or 5×10⁴ to about 5×10⁵ dynes/cm².

In one embodiment, a depot comprises an adherent gel comprising bupivacaine and an anti-inflammatory agent that is evenly distributed throughout the gel. The gel may be of any suitable type, as previously indicated, and should be sufficiently viscous so as to prevent the gel from migrating from the targeted delivery site once deployed; the gel should, in effect, “stick” or adhere to the targeted tissue site. The targeted delivery system may be, for example, a syringe, a catheter, needle or cannula or any other suitable device. The targeted delivery system may inject the gel into or on the targeted tissue site. The therapeutic agent may be mixed into the gel prior to the gel being deployed at the targeted tissue site. In various embodiments, the gel may be part of a two-component delivery system and when the two components are mixed, a chemical process is activated to form the gel and cause it to stick or to adhere to the target tissue.

In various embodiments, a gel is provided that hardens or stiffens after delivery. Typically, hardening gel formulations may have a pre-dosed modulus of elasticity in the range of about 1×10⁴ to about 3×10⁵ dynes/cm², or 2×10⁴ to about 2×10⁵ dynes/cm², or 5×10⁴ to about 1×10⁵ dynes/cm². The post-dosed hardening gels (after delivery) may have a rubbery consistency and have a modulus of elasticity in the range of about 1×10⁴ to about 2×10⁶ dynes/cm², or 1×10⁵ to about 7×10⁵ dynes/cm², or 2×10⁵ to about 5×10⁵ dynes/cm².

In various embodiments, for those gel formulations that contain a polymer, the polymer concentration may affect the rate at which the gel hardens (e.g., a gel with a higher concentration of polymer may coagulate more quickly than gels having a lower concentration of polymer). In various embodiments, when the gel hardens, the resulting matrix is solid but is also able to conform to the irregular surface of the tissue (e.g., recesses and/or projections in bone). In other various embodiments, the gel will not harden upon tissue contact after being injected to the tissue site.

The percentage of polymer present in the gel may also affect the viscosity of the polymeric composition. For example, a composition having a higher percentage by weight of polymer is typically thicker and more viscous than a composition having a lower percentage by weight of polymer. A more viscous composition tends to flow more slowly. Therefore, a composition having a lower viscosity may be preferred in some instances.

In various embodiments, the molecular weight of the gel can be varied by many methods known in the art. The choice of method to vary molecular weight is typically determined by the composition of the gel (e.g., polymer, versus non-polymer). For example in various embodiments, when the gel comprises one or more polymers, the degree of polymerization can be controlled by varying the amount of polymer initiators (e.g. benzoyl peroxide), organic solvents or activator (e.g. DMPT), crosslinking agents, polymerization agent, and/or reaction time.

Suitable gel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric gel that includes a high molecular weight polymer, tends to coagulate or solidify more quickly than a polymeric composition that includes a low-molecular weight polymer. Polymeric gel formulations that include high molecular weight polymers, also tend to have a higher solution viscosity than polymeric gels that includes low-molecular weight polymers.

When the gel is designed to be a flowable gel, it can vary from low viscosity, similar to that of water, to a high viscosity, similar to that of a paste, depending on the molecular weight and concentration of the polymer used in the gel. The viscosity of the gel can be varied such that the polymeric composition can be applied to a patient's tissues by any convenient technique, for example, by spraying, brushing, dripping, injecting, or painting. Different viscosities of the gel will depend on the technique used to apply the composition.

In various embodiments, the gel has an inherent viscosity (abbreviated as “I.V.” and units are in deciliters/gram), which is a measure of the gel's molecular weight and degradation time (e.g., a gel with a high inherent viscosity has a higher molecular weight and longer degradation time). Typically, a gel with a high molecular weight provides a stronger matrix and the matrix takes more time to degrade. In contrast, a gel with a low molecular weight degrades more quickly and provides a softer matrix. In various embodiments, the gel has a molecular weight, as shown by the inherent viscosity, from about 0.10 dL/g to about 1.2 dL/g or from about 0.10 dL/g to about 0.40 dL/g.

In various embodiments, the gel can have a viscosity of about 300 to about 5,000 centipoise (cp). In other embodiments, the gel can have a viscosity of from about 5 to about 300 cps, from about 10 cps to about 50 cps, from about 15 cps to about 75 cps at room temperature. The gel may optionally have a viscosity enhancing agent such as, for example, hydroxypropyl cellulose, hydroxypropyl methylcellulose, hydroxyethyl methylcellulose, carboxymethylcellulose and salts thereof, Carbopol, poly-(hydroxyethylmethacrylate), poly(methoxyethylmethacrylate), poly-(methoxyethoxyethyl methacrylate), polymethylmethacrylate (PMMA), methylmethacrylate (MMA), gelatin, polyvinyl alcohols, propylene glycol, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

A gel with a higher viscosity may be desirable for certain applications, for example, a gel having a putty-like consistency may be more preferable for bone regeneration applications. In various embodiments, when a polymer is employed in the gel, the polymeric composition includes about 10 wt % to about 90 wt % or about 30 wt % to about 60 wt % of the polymer.

In various embodiments, the gel is a hydrogel made of high molecular weight biocompatible elastomeric polymers of synthetic or natural origin. A desirable property for the hydrogel to have is the ability to respond rapidly to mechanical stresses, particularly shears and loads, in the human body.

Hydrogels obtained from natural sources are particularly appealing because they are more likely to be biodegradable and biocompatible for in vivo applications. Suitable hydrogels include natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly (acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof.

In various embodiments, rather than directly admixing the therapeutic agents into the gel, bupivacaine and anti-inflammatory agent loaded polymer microspheres may be dispersed within the gel. In one embodiment, the microspheres provide for a sustained release of both bupivacaine and an anti-inflammatory agent. The bupivacaine and anti-inflammatory agent may occupy the same or different microspheres. In yet another embodiment, a biodegradable gel prevents the microspheres from releasing the bupivacaine and anti-inflammatory agent; the microspheres thus do not release the bupivacaine and anti-inflammatory agent until the microspheres themselves have been released from the gel. For example, a gel may be deployed around a target tissue site (e.g., a nerve root), thus allowing the drug loaded microspheres to deliver drug directly to the point of interest.

Microspheres, much like a fluid, may disperse relatively quickly, depending upon the surrounding tissue type, and hence disperse the bupivacaine and anti-inflammatory agent. In some situations, this may be desirable; in others, it may be more desirable to keep the bupivacaine and anti-inflammatory agent tightly constrained to a well-defined target site. The present invention also contemplates the use of adherent gels to so constrain dispersal of the therapeutic agent. These gels may be deployed, for example, in a disc space, in a spinal canal, or in surrounding tissue.

It will be appreciated by those with skill in the art that the depot can be administered to the target site using a “cannula” or “needle” that can be a part of a drug delivery device e.g., a syringe, a gun drug delivery device, or any medical device suitable for the application of a drug to a targeted organ or anatomic region. The cannula or needle of the drug depot device is designed to cause minimal physical and psychological trauma to the patient.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. In various embodiments, the cannula or needle may also be non-coring and have a sheath covering it to avoid unwanted needle sticks.

The preferred dimensions of the hollow cannula or needle, among other things, will depend on the site for implantation. For example, the width of the epidural space is only about 3-5 mm for the thoracic region and about 5-7 mm for the lumbar region. Thus, the needle or cannula, in various embodiments, can be designed for these specific areas. In various embodiments, the cannula or needle may be inserted using a transforaminal approach in the spinal foramen space, for example, along an inflamed nerve root and the drug depot implanted at this site for treating the condition. Typically, the transforaminal approach involves approaching the intervertebral space through the intervertebral foramina.

Some examples of lengths of the cannula or needle may include, but are not limited to, from about 50 to 150 mm in length, for example, about 65 mm for epidural pediatric use, about 85 mm for a standard adult and about 110 mm for an obese adult patient. The thickness of the cannula or needle will also depend on the site of implantation. In various embodiments, the thickness includes, but is not limited to, from about 0.05 mm to about 1.655 mm. The gauge of the cannula or needle may be the widest or smallest diameter or a diameter in between for insertion into a human or animal body. The widest diameter is typically about 14 gauge, while the smallest diameter is about 25 gauge. In various embodiments the gauge of the needle or cannula is about 18 to about 22 gauge.

In various embodiments, like the drug depot and/or gel, the cannula or needle includes dose radiographic markers that indicate location at or near the site beneath the skin, so that the user may accurately position the depot at or near the site using any of the numerous diagnostic imaging procedures. Such diagnostic imaging procedures include, for example, X-ray imaging or fluoroscopy. Examples of such radiographic markers include, but are not limited to, barium, calcium phosphate, and/or metal beads or particles.

In various embodiments, the needle or cannula may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of the needle or cannula relative to the absence of the material or topography.

The drug depot, and/or medical device to administer the drug may be sterilizable. In various embodiments, one or more components of the drug depot, and/or medical device to administer the drug are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy that is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the drug depot is included in a gel.

Other methods may also be used to sterilize the depot and/or one or more components of the device, including, but not limited to, gas sterilization, such as, for example, with ethylene oxide or steam sterilization.

Kit

In various embodiments, a kit is provided that may include additional parts along with the drug depot and/or medical device combined together to be used to implant the drug depot (e.g., ribbon-like fibers). The kit may include the drug depot device in a first compartment. The second compartment may include a canister holding the drug depot and any other instruments needed for the localized drug delivery. A third compartment may include gloves, drapes, wound dressings and other procedural supplies for maintaining sterility of the implanting process, as well as an instruction booklet. A fourth compartment may include additional cannulas and/or needles. A fifth compartment may include an agent for radiographic imaging. Each tool may be separately packaged in a plastic pouch that is radiation sterilized. A cover of the kit may include illustrations of the implanting procedure and a clear plastic cover may be placed over the compartments to maintain sterility.

Administration

In various embodiments, the drug depot containing the active ingredient(s) may be parenterally administered. In addition to including administration that is intravenous, intramuscular, through continuous or intermittent infusion, intraperitoneal, intrasternal, subcutaneous, intra-operatively, intrathecally, intradiskally, peridiskally, epidurally, perispinally, intra-articularly or a combination thereof, parenteral administration also includes an infusion pump that administers a pharmaceutical composition through a catheter near the target site, an implantable mini-pump that can be inserted at or near the target site, and/or an implantable controlled release device or sustained release delivery system that can release a certain amount of the composition per hour or in intermittent bolus doses.

One example of a suitable pump for use is the SynchroMed® (Medtronic, Minneapolis, Minn.) pump. The pump has three sealed chambers. One contains an electronic module and battery. The second contains a peristaltic pump and drug reservoir. The third contains an inert gas that provides the pressure needed to force the pharmaceutical composition into the peristaltic pump. To fill the pump, the pharmaceutical composition is injected through the reservoir fill port to the expandable reservoir. The inert gas creates pressure on the reservoir, and the pressure forces the pharmaceutical composition through a filter and into the pump chamber. The pharmaceutical composition is then pumped out of the device from the pump chamber and into the catheter, which will direct it for deposit at the target site. The rate of delivery of pharmaceutical composition is controlled by a microprocessor. This allows the pump to be used to deliver similar or different amounts of pharmaceutical composition continuously, at specific times, or at set intervals.

Potential drug delivery devices suitable for adaptation for the methods described herein include but are not limited to those described, for example, in U.S. Pat. No. 6,551,290 (assigned to Medtronic, the entire disclosure of which is herein incorporated by reference), which describes a medical catheter for target specific drug delivery; U.S. Pat. No. 6,571,125 (assigned to Medtronic, the entire disclosure of which is herein incorporated by reference), which describes an implantable medical device for controllably releasing a biologically active agent; U.S. Pat. No. 6,594,880 (assigned to Medtronic, the entire disclosure of which is herein incorporated by reference), which describes an interparenchymal infusion catheter system for delivering therapeutic agents to selected sites in an organism; and U.S. Pat. No. 5,752,390 (assigned to Medtronic, the entire disclosure of which is herein incorporated by reference), which describes an implantable catheter for infusing equal volumes of agents to spaced sites. In various embodiments, pumps may be adapted with a pre-programmable implantable apparatus with a feedback regulated delivery, a micro-reservoir osmotic release system for controlled release of chemicals, small, light-weight devices for delivering liquid medication, implantable micro-miniature infusion devices, implantable ceramic valve pump assemblies, or implantable infusion pumps with a collapsible fluid chamber. Alzet® osmotic pumps (Durect Corporation, Cupertino, Calif.) are also available in a variety of sizes, pumping rates, and durations suitable for use in the described methods.

In various embodiments, a method for delivering a therapeutic agent into a surgery site of a patient is provided, the method comprising inserting a cannula at or near a target tissue site and implanting the drug depot at the target site beneath the skin of the patient and brushing, dripping, injecting, or painting the gel in the target site to hold or have the drug depot adhere to the target site. In this way unwanted migration of the drug depot away from the target site is reduced or eliminated.

In various embodiments, to administer the gel having the drug depot dispersed therein to the desired site, first the cannula or needle can be inserted through the skin and soft tissue down to the target tissue site and the gel administered (e.g., brushed, dripped, injected, or painted, etc.) at or near the target site. In those embodiments where the drug depot is separate from the gel, first the cannula or needle can be inserted through the skin and soft tissue down to the site of injection and one or more base layer(s) of gel can be administered to the target site. Following administration of the one or more base layer(s), the drug depot can be implanted on or in the base layer(s) so that the gel can hold the depot in place or reduce migration. If required a subsequent layer or layers of gel can be applied on the drug depot to surround the depot and further hold it in place. Alternatively, the drug depot may be implanted first and then the gel placed (e.g., brushed, dripped, injected, or painted, etc.) around the drug depot to hold it in place. By using the gel, accurate and precise implantation of a drug depot can be accomplished with minimal physical and psychological trauma to the patient. The gel also avoids the need to suture the drug depot to the target site reducing physical and psychological trauma to the patient.

In various embodiments, when the target site comprises a spinal region, a portion of fluid (e.g., spinal fluid, etc.) can be withdrawn from the target site through the cannula or needle first and then the depot administered (e.g., placed, dripped, injected, or implanted, etc.). The target site will re-hydrate (e.g., replenishment of fluid) and this aqueous environment will cause the drug to be released from the depot.

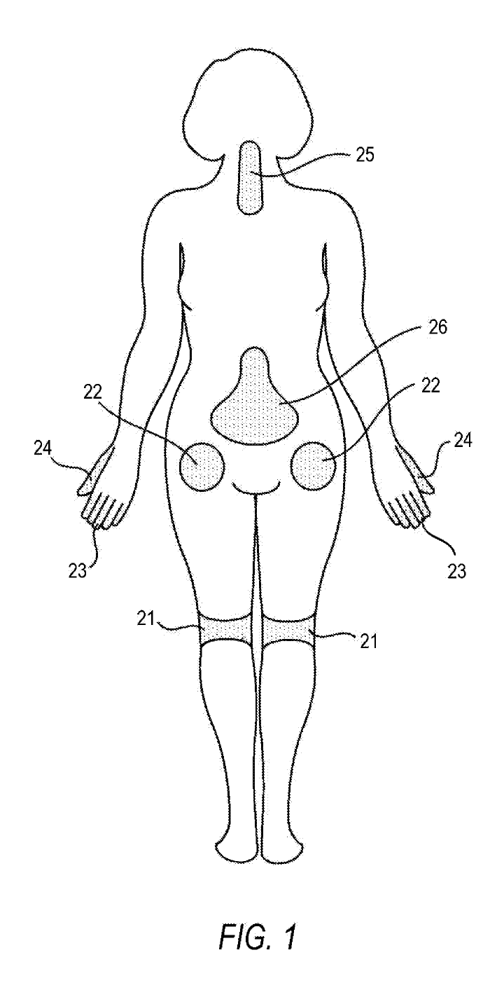

FIG. 1 illustrates a number of common locations within a patient that may be sites at which surgery can take place. It will be recognized that the locations illustrated in FIG. 1 are merely exemplary of the many different locations within a patient that may be at which surgery can take place. For example, surgery may be required at a patient's knees 21, hips 22, fingers 23, thumbs 24, neck 25, and spine 26. Thus, during or following these surgeries, the patient may be subject to pain and require pain management medication.

The term “pain management medication” includes one or more therapeutic agents that are administered to prevent, alleviate or remove pain entirely. These include anti-inflammatory agents, muscle relaxants, analgesics, anesthetics, narcotics, and so forth, and combinations thereof.

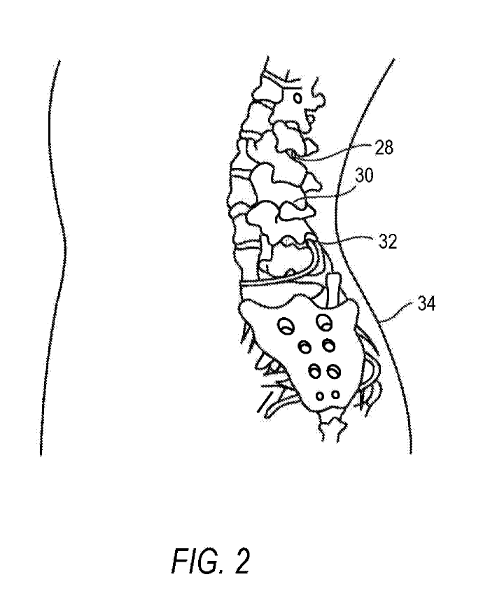

One exemplary embodiment where the depot is suitable for use in post-operative pain as illustrated in FIG. 2. Schematically shown in FIG. 2 is a dorsal view of the spine and sites where the drug depot may be inserted using a cannula or needle beneath the skin 34 to a spinal site 32 (e.g., spinal disc space, spinal canal, soft tissue surrounding the spine, nerve root, etc.) and one or more drug depots 28 and 32 are delivered to various sites along the spine. In this way, when several drug depots are to be implanted, they are implanted in a manner that optimizes location, accurate spacing, and drug distribution.

Although the spinal site is shown, as described above, the drug depot can be delivered to any site beneath the skin, including, but not limited to, at least one muscle, ligament, tendon, cartilage, spinal disc, spinal foraminal space, near the spinal nerve root, or spinal canal.

In some embodiments, it is preferable to co-administer bupivacaine and an anti-inflammatory agent with an antagonist to counteract undesirable effects, for example the compounds such as 5-fluorodeoxyuridine (FUDR) and 3,4 dehydroprolene may also be included. These compounds may prevent or reduce glial and fibroblastic scar formation associated with some types of surgeries.

The bupivacaine and anti-inflammatory-based formulation described herein may be used as medicaments in the form of pharmaceutical preparations. The preparations may be formed with a suitable pharmaceutical carrier that may be solid, semi-solid or liquid, and placed in the appropriate form for parenteral or other administration as desired. As persons of ordinary skill are aware, known carriers include but are not limited to water, gelatine, lactose, starches, stearic acid, magnesium stearate, sicaryl alcohol, talc, vegetable oils, benzyl alcohols, gums, waxes, propylene glycol, polyalkylene glycols and other known carriers for medicaments.

Another embodiment provides a method for treating a mammal suffering from pain associated with surgery, said method comprising administering a therapeutically effective amount of bupivacaine and an anti-inflammatory agent at a target site beneath the skin to relax muscle at or near the target site. The bupivacaine and an anti-inflammatory agent may for example be administered locally to the target tissue site as a drug depot. The term “locally” refers to a proximity to the site of interest such that when the drug is released, an effective amount of the bupivacaine and an anti-inflammatory agent will reach the site.

In some embodiments, the therapeutically effective dosage amount and the release rate profile are sufficient to treat the post-operative pain or disease or condition for a period of 3-14 days; in other embodiments the release rate profile is sufficient to treat for a period of 7-10 days.

In some embodiments, the bupivacaine and an anti-inflammatory agent are encapsulated in a plurality of depots comprising microparticles, microspheres, microcapsules, and/or microfibers. The active ingredients may be combined and then encapsulated or first encapsulated and then combined.

In some embodiments there is a composition useful for the treatment of post-operative pain comprising an effective amount of bupivacaine and an anti-inflammatory agent that is capable of being administered to a post-operative surgery site.

In various embodiments, where the target tissue site comprises blood vessels, a vasoconstrictor may be employed either in or in connection with the drug depot. When the vasoconstrictor is released, it lengthens the duration of an anesthetic response and reduces the systemic uptake of an anesthetic agent, such as bupivacaine. Exemplary vasoconstrictors include but are not limited to catecholamines e.g., epinephrine, norepinephrine and dopamine, as well as, e.g., metaraminol, phenylephrine, methoxamine, mephentermine, methysergide, ergotamine, ergotoxine, dihydroergotamine, sumatriptan and analogs, and alpha-1 and alpha-2 adrenergic agonists, such as, e.g., guanfacine, guanabenz and dopa (i.e., dihyrdoxyphenylalanine), methyldopa, ephedrine, amphetamine, methamphetamine, methylphenidate, ethylnorepinephrine ritalin, pemoline and other sympathomimetic agents, including active metabolites, derivatives and mixtures of any of the foregoing.

In some embodiments, the bupivacaine and an anti-inflammatory agent are administered parenterally, e.g., by injection. In some embodiments, the injection is intrathecal, which refers to an injection into the spinal canal (intrathecal space surrounding the spinal cord). An injection may also be into a muscle or other tissue. In other embodiments, an anti-inflammatory agent and bupivacaine is administered by placement into an open patient cavity during surgery itself.

In some embodiments, the present invention provides a medicinal composition comprising: (a) a therapeutically effective amount of bupivacaine or a pharmaceutically acceptable salt thereof; and (b) a therapeutically effective amount of an anti-inflammatory agent or a pharmaceutically acceptable salt thereof. The medicinal compound made further comprise a polymer, e.g., poly(lactic-co-glycolic acid), which is also known as poly(lactide-co-glycolide).

In various embodiments, the molecular weight of the polymer can be a wide range of values. The average molecular weight of the polymer can be from about 1,000 to about 10,000,000; or about 1,000 to about 1,000,000; or about 5,000 to about 500,000; or about 10,000 to about 100,000; or about 20,000 to 50,000.

As persons of ordinary skill in the art are aware, an implantable depot compositions having a blend of polymers with different end groups are used the resulting formulation will have a lower burst index and a regulated duration of delivery. For example, one may use polymers with acid (e.g., carboxylic acid) and ester end groups (e.g., methyl or ethyl ester end groups).

Additionally, by varying the comonomer ratio of the various monomers that form a polymer (e.g., the L/G (lactic acid/glycolic acid) or G/CL (glycolic acid/polycaprolactone) ratio for a given polymer) there will be a resulting depot composition having a regulated burst index and duration of delivery. For example, a depot composition having a polymer with a L/G ratio of 50:50 may have a short duration of delivery ranging from about two days to about one month; a depot composition having a polymer with a L/G ratio of 65:35 may have a duration of delivery of about two months; a depot composition having a polymer with a L/G ratio of 75:25 or L/CL ratio of 75:25 may have a duration of delivery of about three months to about four months; a depot composition having a polymer ratio with a L/G ratio of 85:15 may have a duration of delivery of about five months; a depot composition having a polymer with a L/CL ratio of 25:75 or PLA may have a duration of delivery greater than or equal to six months; a depot composition having a terpolymer of CL/G/L with G greater than 50% and L greater than 10% may have a duration of delivery of about one month and a depot composition having a terpolymer of CL/G/L with G less than 50% and L less than 10% may have a duration months up to six months. In general, increasing the G content relative to the CL content shortens the duration of delivery whereas increasing the CL content relative to the G content lengthens the duration of delivery. Thus, among other things, depot compositions having a blend of polymers having different molecular weights, end groups and comonomer ratios can be used to create a depot formulation having a lower initial burst and a regulated duration of delivery.

In various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

A formulation of the active ingredients of bupivacaine and an anti-inflammatory agent, in combination with a suitable polymer (e.g., PLG) may be malleable and can be extruded into ribbon-like dosage form. In some embodiments, the formulation is implantable into a surgical site at the time of surgery. The active ingredients may then be released from the depot via diffusion in a sustained fashion over a period of time, e.g., 3-12 days, 5-10 days or 7-10 days post surgery in order to provide pain control.

In some embodiments, the present invention is directed to a method of treating or preventing postoperative pain or inflammation in a patient in need of such treatment, the method comprising administering one or more biodegradable drug depots comprising a therapeutically effective amount of bupivacaine or pharmaceutically acceptable salt thereof and an anti-inflammatory agent or pharmaceutically acceptable salt thereof to a target tissue site beneath the skin, wherein the drug depot releases an effective amount of bupivacaine or pharmaceutically acceptable salt thereof and an anti-inflammatory agent or pharmaceutically acceptable salt thereof over a period of 3 to 12 days or 5 to 10 days.

In some embodiments of the present invention, the drug depot may release 5%, 10%, 15%, 20%, 25%, 30%, 40%, 50%, 60%, 70%, 80%, 90%, 95%, or 99% of the bupivacaine or pharmaceutically acceptable salt thereof and an anti-inflammatory agent or pharmaceutically acceptable salt thereof relative to a total amount of bupivacaine or pharmaceutically acceptable salt thereof and an anti-inflammatory agent or pharmaceutically acceptable salt thereof loaded in the drug depot over a period of 3 to 14 days after the drug depot is administered to the target tissue site.

In some embodiments of the present invention, the drug depot releases 5 mg to 60 mg of bupivacaine or pharmaceutically acceptable salt thereof and 10 μg to 100 μg of an anti-inflammatory agent or pharmaceutically acceptable salt thereof every 4 to 6 hours to treat postoperative pain or inflammation over a span of 3 to 14 days or 5 to 12 days or 7 to 10 days.

By way of non-limiting example, the target tissue site comprises at least one muscle, ligament, tendon, cartilage, spinal disc, spinal foraminal space near the spinal nerve root, facet or spinal canal. By way of further example, the surgery may be arthroscopic surgery, an excision of a mass, hernia repair, spinal fusion, thoracic, cervical, or lumbar surgery, pelvic surgery or a combination thereof.

In some embodiments of the present invention, the bupivacaine or pharmaceutically acceptable salt thereof and an anti-inflammatory agent or pharmaceutically acceptable salt thereof is encapsulated in a plurality of depots comprising microparticles, microspheres, microcapsules, and/or microfibers suspended in a gel.

In some embodiments, the drug depot further comprises a radiographic marker adapted to assist in radiographic imaging. The radiographic marker may for example, comprise barium, calcium phosphate, and/or metal beads.

In some embodiments, the present invention provides a method of inhibiting postoperative pain or inflammation in a patient in need of such treatment, the method comprising delivering one or more biodegradable drug depots comprising a therapeutically effective amount of bupivacaine or pharmaceutically acceptable salt thereof and an anti-inflammatory agent or pharmaceutically acceptable salt thereof to a target tissue site beneath the skin before, during or after surgery, wherein the drug depot releases an effective amount of bupivacaine or pharmaceutically acceptable salt thereof and an anti-inflammatory agent or pharmaceutically acceptable salt thereof over a period of 3 to 14 days or 5 to 12 days.

In some embodiments, the present invention provides a method of inhibiting postoperative pain and undesirable levels of inflammation, wherein the drug depot (i) releases 2 mg to 60 mg of bupivacaine or pharmaceutically acceptable salt thereof and 1 to 4 μg an anti-inflammatory agent every 4 to 6 hours to inhibit postoperative pain or inflammation. The drug depot may further comprise at least one anabolic or an anti-catabolic growth factor or combination thereof.

In some embodiments, the present invention provides an implantable drug depot useful for preventing or treating postoperative pain or inflammation in a patient in need of such treatment, the implantable drug depot comprising a therapeutically effective amount of bupivacaine or pharmaceutically acceptable salt thereof and an anti-inflammatory agent, the depot being implantable at a site beneath the skin to prevent or treat postoperative pain, wherein the drug depot releases an effective amount of bupivacaine or pharmaceutically acceptable salt thereof and an anti-inflammatory agent or pharmaceutically acceptable salt thereof over a period of 3 to 14 days or 5 to 12 days.

In some embodiments, the present invention provides an implantable drug depot, wherein the drug depot (i) comprises one or more immediate release layer(s) that releases a bolus dose of bupivacaine or pharmaceutically acceptable salt thereof and an anti-inflammatory agent or pharmaceutically acceptable salt thereof at a site beneath the skin and (ii) one or more sustain release layer(s) that releases an effective amount of bupivacaine or pharmaceutically acceptable salt thereof and an anti-inflammatory agent an anti-inflammatory agent or pharmaceutically acceptable salt thereof over a period of 3 to 14 days or 5 to 12 days. By way of example, in the drug depot, the one or more immediate release layer(s) may comprise poly (lactide-co-glycolide) (PLGA) and the one or more sustain release layer(s) may comprise polylactide (PLA).

Compounding

In some embodiments, the anti-inflammatory is first compounded with a polymer to make a first component of the drug depot. In this first component, the anti-inflammatory may for example, comprise 10% to 20% by weight. The bupivacaine may separately be compounded with a polymer to make a second component of the drug depot. In this second component, the bupivacaine may for example comprises 50%-70% by weight. In some embodiments, the percentage of bupivacaine to anti-inflammatory is between 1:1 and 10:1. In some embodiments, the percentage of bupivacaine to anti-inflammatory is between 2:1 and 10:1. In some embodiments, the percentage of bupivacaine to anti-inflammatory is between 3:1 and 10:1. In some embodiments, the percentage of bupivacaine to anti-inflammatory is between 4:1 and 10:1. In some embodiments, the percentage of bupivacaine to anti-inflammatory is between 5:1 and 10:1. In some embodiments, the percentage of bupivacaine to anti-inflammatory is between 6:1 and 10:1. In some embodiments, the percentage of bupivacaine to anti-inflammatory is between 7:1 and 10:1. In some embodiments, the percentage of bupivacaine to anti-inflammatory is between 8:1 and 10:1. In some embodiments, the percentage of bupivacaine to anti-inflammatory is between 9:1 and 10:1. In some embodiments, the percentage of bupivacaine to anti-inflammatory is between 1:1 and 9:1. In some embodiments, the percentage of bupivacaine to anti-inflammatory is between 1:1 and 8:1. In some embodiments, the percentage of bupivacaine to anti-inflammatory is between 1:1 and 7:1. In some embodiments, the percentage of bupivacaine to anti-inflammatory is between 1:1 and 6:1. In some embodiments, the percentage of bupivacaine to anti-inflammatory is between 1:1 and 5:1. In some embodiments, the percentage of bupivacaine to anti-inflammatory is between 1:1 and 4:1. In some embodiments, the percentage of bupivacaine to anti-inflammatory is between 1:1 and 3:1. In some embodiments, the percentage of bupivacaine to anti-inflammatory is between 1:1 and 2:1.

In some embodiments, the amount of anti-inflammatory released is at least 10%, at least 20%, at least 30%, at least 40%, at least 50%, at least 60%, at least 70%, at least 80%, at least 90% more than the percent of bupivacaine that is released over the first two days. In some embodiments after the first two days, the remaining bupivacaine and the remaining anti-inflammatory are released at approximately the same rate.

The bupivacaine and an anti-inflammatory agent may also be formulated together a two active ingredients with one polymer. Thus, a combination product comprising an anti-inflammatory agent and bupivacaine may by way of example be formed by combining these active ingredients with a polymer as part of one formulation to generate a combination drug product. By way of another example, each active formulation is separately developed for co-administration to a site, e.g., a surgical wound site.

In some embodiments, the amount of bupivacaine is present in an amount sufficient to release between 2 mg/day to 1800 mg/day, and the amount of an anti-inflammatory agent is sufficient to release between 2 and 40 μg/day. In some embodiments, the amount of bupivacaine is present in an amount sufficient to release between 10 and 1500 mg/day, and the amount of an anti-inflammatory agent present is in an amount sufficient to release between 10 and 30 μg/day. The release of each compound may be for at least three, at least four at least five, at least six, at least seven or at least eight days in the recited ranges.

In various embodiments, the drug particle size is from about 5 to 30 micrometers, however, in various embodiments ranges from about 1 micron to 250 microns may be used.

In some embodiments, there is another method of making an implantable drug depot. In this method, one combines a biocompatible polymer and a therapeutically effective amount of bupivacaine and an anti-inflammatory agent and forms the implantable drug depot from the combination.

Processing

Various techniques are available for forming at least a portion of a drug depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise, one or more of the following: bupivacaine and an anti-inflammatory agent and other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: bupivacaine and an anti-inflammatory agent, optional additional therapeutic agent(s), radiographic agent(s), and so forth. The resulting mixture is then shaped into an implantable drug depot. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, bupivacaine and an anti-inflammatory agent may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent under conditions of lower temperature and mechanical shear, and the resulting mixture is shaped into the bupivacaine and an anti-inflammatory agent containing drug depot. Conversely, in another embodiment, the biocompatible polymer can be precompounded with the therapeutic agent under conditions of reduced temperature and mechanical shear. This precompounded material is then mixed with, for example, a radio-opacifying agent, also under conditions of reduced temperature and mechanical shear, and the resulting mixture is shaped into the drug depot.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a depot is formed comprising PLGA or PLA polymer, a radio-opacifying agent (e.g., bismuth subcarbonate), and a therapeutic agent prone to degradation by heat and/or mechanical shear (e.g., bupivacaine and an anti-inflammatory agent), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of bupivacaine and an anti-inflammatory agent, because processing at or above these temperatures will result in substantial therapeutic agent degradation. It is further noted that in certain embodiments, the processing temperature will be below the melting point of all bioactive compounds within the composition, including the therapeutic agent. After compounding, the resulting depot is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing drug depot (the pre-existing drug depot can be formed using a variety of techniques including solution and thermoplastic processing techniques, and it can comprise a variety of additives including a radio-opacifying agent and/or viscosity enhancing agent), whereupon the therapeutic agent is imbibed on or in the drug depot. As above, the resulting solid material can then be granulated for further processing, if desired.

Typically, an extrusion processes may be used to form the drug depot comprising a biocompatible polymer(s), therapeutic agent(s) and radio-opacifying agent(s). Co-extrusion may also be employed, which is a shaping process that can be used to produce a drug depot comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depot that may emerge from the thermoplastic processing (e.g., ribbon, pellet, strip, etc.) is cooled. Examples of cooling processes include air cooling and/or immersion in a cooling bath. In some embodiments, a water bath is used to cool the extruded depot. However, where a water-soluble therapeutic agent such as bupivacaine and certain anti-inflammatory agents are used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot surface, thus controlling or minimizing a high drug dose “initial burst” or “bolus dose” upon implantation or insertion if this is release profile is not desired.

In various embodiments, the drug depot can be prepared by mixing or spraying the drug with the polymer and then molding the depot to the desired shape.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. An implantable drug depot useful for localized delivery to a site beneath the skin of a patient, the drug depot comprising: a therapeutically effective amount of bupivacaine and an anti-inflammatory agent, wherein the drug depot is capable of releasing the bupivacaine and the anti-inflammatory agent over three days to fourteen days where the depot is capable of releasing a first percentage of bupivacaine relative to a total amount of bupivacaine over the first two days and a first percentage of anti-inflammatory agent relative to a total amount of anti-inflammatory agent over the first two days, wherein the first percentage of anti-inflammatory agent is less than the first percentage of bupivacaine.

2. An implantable drug depot of claim 1, wherein the bupivacaine is in the form of a base and the anti-inflammatory agent is an NSAID.

3. An implantable drug depot of claim 1, wherein the bupivacaine is in the form of a salt and the anti-inflammatory agent is an NSAID.

4. An implantable drug depot of claim 1, wherein the bupivacaine is in the form of a base and the anti-inflammatory agent is a COX-2 inhibitor.

5. An implantable drug depot of claim 1, wherein the bupivacaine is in the form of a salt and the anti-inflammatory agent is a COX-2 inhibitor.

6. A method of treating or preventing postoperative pain in a patient in need of such treatment, the method comprising administering one or more biodegradable drug depots comprising the drug depot of claim 1 to a target tissue site beneath the skin, wherein the drug depot releases an effective amount of said bupivacaine and said anti-inflammatory agent over a period of 3 to 14 days.

7. A method of treating or preventing postoperative pain according to claim 6, wherein one or more drug depots release an effective amount of bupivacaine and anti-inflammatory agent over a period of 5 to 12 days.

8. A method of treating or preventing postoperative pain or inflammation according to claim 6, wherein the drug depot releases 50%, 60%, 70%, 80%, 90%, 95%, or 99% of the bupivacaine relative to a total amount of bupivacaine over a period of 3 to 14 days after the drug depot is administered to the target tissue site.

9. A method of treating or preventing postoperative pain according to claim 6, wherein the drug depot releases 5 mg to 60 mg of bupivacaine and 1 μg to 4 μg of anti-inflammatory agent every 4 to 6 hours to treat postoperative pain.

10. A method of treating or preventing postoperative pain according to claim 6, wherein the target tissue site comprises at least one muscle, ligament, tendon, cartilage, spinal disc, spinal foraminal space near the spinal nerve root, facet or synovial joint, or spinal canal.

11. A method of treating or preventing postoperative pain according to claim 6, wherein the drug depot comprises at least one anabolic or an anti-catabolic growth factor or a combination thereof.

12. A method of treating or preventing postoperative pain according to claim 6, wherein the bupivacaine and anti-inflammatory agent is encapsulated in a plurality of depots comprising microparticles, microspheres, microcapsules, and/or microfibers suspended in a gel.

13. A method of treating or preventing postoperative pain according to claim 6, wherein the pain or inflammation is associated with orthopedic surgery, spine surgery, arthroscopic surgery, an excision of a mass, hernia repair, spinal fusion, thoracic, cervical, or lumbar surgery, pelvic surgery or a combination thereof.

14. A method of treating or preventing postoperative pain according to claim 6, wherein the drug depot comprises a radiographic marker adapted to assist in radiographic imaging.

15. A method of treating or preventing postoperative pain according to claim 14, wherein the radiographic marker comprises barium, calcium, and/or metal beads.

16. A method of inhibiting postoperative pain in a patient in need of such treatment, the method comprising delivering one or more biodegradable drug depots comprising the drug depot of claim 1 to a target tissue site beneath the skin before, during or after surgery, wherein the drug depot releases an effective amount of bupivacaine and anti-inflammatory agent over a period of 3 to 14 days.

17. A method of inhibiting postoperative pain according to claim 16, wherein one or more drug depots release an effective amount of bupivacaine and anti-inflammatory agent over a period of 5 to 12 days.

18. A method of inhibiting postoperative pain according to claim 17, wherein the drug depot releases 5%, 10%, 15%, 20%, 25%, 30%, 40%, 50%, 60%, 70%, 80%, 90%, 95%, or 99% of bupivacaine relative to a total amount of bupivacaine loaded in the drug depot over a period of 3 to 14 days after the drug depot is administered to the target tissue site.

19. A method of treating or preventing postoperative pain or inflammation in a patient in need of such treatment, the method comprising co-administering a first biodegradable drug depot and a second biodegradable drug depot to a target site, wherein said first biodegradable drug depot releases an effective amount of bupivacaine over a period of 3 to 14 days and wherein said second biodegradable drug depot releases an anti-inflammatory agent over a period of 3 to 14 days.

20. An implantable drug depot useful for localized delivery to a site beneath the skin of a patient, the drug depot comprising: a therapeutically effective amount of bupivacaine and an anti-inflammatory agent, wherein the drug depot is capable of releasing the bupivacaine and the anti-inflammatory agent over five days to twelve days and wherein the drug depot is capable of releasing a first percentage of bupivacaine relative to a total amount of bupivacaine over the first two days and a first percentage of anti-inflammatory agent relative to a total amount of anti-inflammatory agent over the first two days, wherein the first percentage of bupivacaine is greater than the first percentage of the anti-inflammatory agent and wherein the anti-inflammatory agent is a COX-2 inhibitor.
